(19) United States
(12) Patent Application Publication
(10) Pub. No.: US 2011/0130540 A1
(43) Pub. Date: Jun. 2, 2011

(54) PROCESSES FOR THE SYNTHESIS OF 3-HYDROXYGLUTARONITRILE

(75) Inventor: Jelena Cirakovic, Wilmington, DE (US)

(73) Assignee: E.I. DU PONT DE NEMOURS AND COMPANY, Wilmington, DE (US)

(21) Appl. No.: 12/672,724

(22) PCT Filed: Aug. 14, 2008
(86) PCT No.: PCT/US08/73134

§ 371 Date: Feb. 9, 2010

Related U.S. Application Data
(60) Provisional application No. 60/956,501, filed on Aug. 17, 2007.

(51) Int. Cl. C07C 291/10; C07D 213/57; C08G 73/06

(52) U.S. Cl. 528/423; 558/302; 546/330

(57) ABSTRACT

There are disclosed high yield and high productivity processes for preparing 3-hydroxyglutaronitrile by reacting allyl cyanide epoxide with a basic aqueous solution of a cyanide source.

This application claims priority under 35 U.S.C. §119(e) from, and claims the benefit of, U.S. Provisional Application No. 60/956,501, filed Aug. 17, 2007, which is by this reference incorporated in its entirety as a part hereof for all purposes.

### TECHNICAL FIELD

This invention relates to the manufacture of 3-hydroxyglutaronitrile, which is a useful intermediate in chemical synthesis.

### BACKGROUND

The compound 3-hydroxyglutaronitrile is a precursor for a variety of useful materials such as pharmaceutically active compounds, diamines used in hair coloring, and monomers for high-strength fibers. It has conventionally been synthesized by treating epichlorohydrin (“ECH”) with an inorganic cyanide in water, producing 4-chloro-3-hydroxy-butanenitrile (also known as “chlorohydrin”) as an intermediate, as shown for example by F. Johnson et al, J. Org. Chem. (1962), 27, 2241-2243:

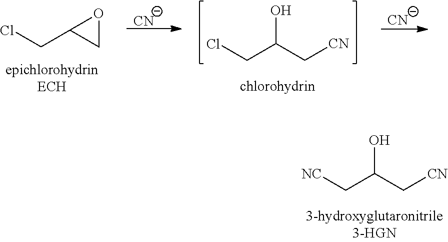

This process suffers, however, from byproduct formation and levels of productivity that are not commercially advantageous.

U.S. Provisional Application No. 60/874,401, which is by this reference incorporated in its entirety as a part hereof for all purposes, provides high yield and high productivity processes for preparing 3-hydroxyglutaronitrile by reacting an epihalohydrin, a 4-halo-3-hydroxy-butanenitrile, or a compound analogous to those materials in which the leaving group is other than a halogen, with cyanide (CN⁻) in the presence of water and an ionic liquid. This reaction has a useful level of yield, and observed reaction times to completion are from about 17 to about 48 hours.

A need remains, however, for other processes by which 3-hydroxyglutaronitrile can be manufactured with high yield and short reaction time.

### SUMMARY

The inventions disclosed herein include processes for the preparation of 3-hydroxyglutaronitrile, processes for the preparation of products into which 3-hydroxyglutaronitrile can be converted, and the products obtained and obtainable by all such processes.

One embodiment of these processes provides a process in which 3-hydroxyglutaronitrile is prepared by

(a) epoxidizing a solution of allyl cyanide to form allyl cyanide epoxide, which is represented by the structure of Formula I as follows:

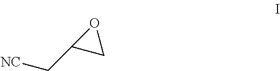

and

(b) contacting the allyl cyanide epoxide with an aqueous solution of a CN— source that has a pH in the range of about 8 to about 10.

Another embodiment of these processes provides a process in which 3-hydroxyglutaronitrile is converted to another compound, or to an oligomer or a polymer.

### DETAILED DESCRIPTION

In a process hereof, 3-hydroxyglutaronitrile (“3-HGN”) is produced by forming the epoxide of allyl cyanide, and then contacting the allyl cyanide epoxide so formed with a basic aqueous solution of a cyanide source. 3-HGN (CAS Registry No. 624-58-8) is also known as oxiraneacetonitrile, 3,4-epoxybutyronitrile and epicyanohydrin, and is also represented by the structure of Formula I.

In the first step of a process hereof, the epoxide of allyl cyanide is formed. Allyl cyanide (CH₂═CHCH₂CN, CAS Reg. No. 109-75-1, also known as 3-butenenitrile) is commercially available, e.g. from Sigma-Aldrich (St. Louis, Mo., USA). The epoxidation of allyl cyanide is described, for example, in F. F. Fleming et al, Journal of Organic Chemistry 66, pp. 2174-2177 (2001); and E. Mete et al, Russian Chemical Bulletin, International Edition, 52(8), pp. 1879-1881 (August, 2003).

Fleming added 1.5 equivalents solid m-chloroperbenzoic acid (“mCPBA”) to a room temperature solution of allyl cyanide in CH₂Cl₂ (0.1-0.5 M). One-eighth of the mCPBA was added each day for a total of eight days. The resultant solution was stirred overnight, after which saturated, aqueous NaHSO₃ was added to reduce the excess mCPBA to m-chlorobenzoic acid (“mCBA”). To isolate the product, the organic phase was separated, washed with saturated aqueous NaHCO₃ and then dried over anhydrous Na₂SO₄ or MgSO₄, and concentrated under reduced pressure to produce analytically pure allyl cyanide epoxide. Mete similarly reacted allyl cyanide with mCPBA (one equivalent, in CH₂Cl) but also sonicated the reaction mixture in an ultrasonic bath (47 kHz) for two days to expedite the reaction.

In an alternative embodiment, a process hereof includes a step of isolating the allyl cyanide epoxide as produced by the process as described above.

3-HGN is then formed from allyl cyanide epoxide, the reaction for which may be represented schematically as follows:

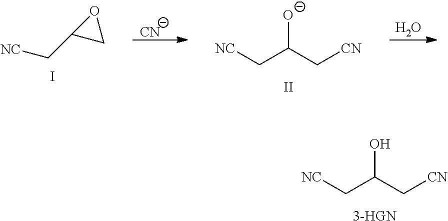

Allyl cyanide epoxide is contacted with an aqueous solution of a cyanide source. A suitable aqueous solution of a cyanide source contains about 1 to about 1.5, preferably about 1.1 to about 1.3, moles of CN⁻ for each mole of allyl cyanide epoxide with which it is to be contacted. Suitable CN⁻ sources include without limitation alkali cyanides such as KCN, NaCN and LiCN; and trimethylsilyl cyanide. Acetone cyanohydrin may be used, in which case a base such as triethylamine is added with it in relative amounts such that more than one mole of acetone cyanohydrin is added per mole of base, or about 3 to about 4 moles of acetone cyanohydrin are added per mole of base.

The pH of the aqueous solution of cyanide source may be about 8.0 or more, about 8.3 or more, about 8.7 or more, or about 9.0 or more, and yet about 10.0 or less, about 9.7 or less, about 9.3 or less, or about 9.0 or less. The pH of the aqueous solution of cyanide source may thus be expressed as any of the possible ranges that may be formed by any combination of the various maxima and minima, as set forth above. A pH of about 8.0 or more is preferred. An aqueous solution of cyanide source at the desired pH may be provided by adjusting the pH of an aqueous cyanide solution by adding enough acid thereto to lower the pH to the range of about 8.0 to about 10.0. The specific acid used for this purpose is not critical. Examples of suitable acids include without limitation H₂SO₄ and HCl.

Allyl cyanide epoxide (Formula I) is contacted with an aqueous solution of cyanide source to obtain the reaction thereof for a time sufficient to produce a 3-HGN product via the intermediate described generally above by Formula II, a sufficient time being, for example, about 4 to about 10 hours. The aqueous solution of cyanide source as used in such reaction may suitably have a temperature in the range, for example, of about 0 to about 25° C.

After a time sufficient for product formation, the reaction mixture is allowed to separate into organic and aqueous layers to allow isolation of the 3-HGN product. In general, the 3-HGN product resides largely in the aqueous phase, and the water layer may thus be extracted, for example, with ethyl acetate, tetrahydrofuran (“THF”), cyclopentanone, cyclohexanone, or methylethylketone (“MEK”). The organic extracts are concentrated, and the residue is purified by any suitable means known in the art, such as column chromatography, to yield the product 3-HGN as a yellow oil.

The 3-HGN product may, as desired, be isolated and recovered as described above. It may also be subjected with or without recovery from the reaction mixture to further steps to convert it to another product such as another compound (e.g. a monomer), or an oligomer or a polymer. Another embodiment of a process hereof thus provides a process for converting 3-HGN, through one or more reactions, into another compound, or into an oligomer or a polymer. 3-HGN may be made by a process such as described above, and then converted, for example, into a compound such as a diaminopyridine. In a multi-step process, a diaminopyridine may in turn be subjected to a polymerization reaction to prepare an oligomer or polymer therefrom, such as those having amide functionality, imide functionality, or urea functionality, or a pyridobisimidazole-2,6-diyl(2,5-dihydroxy-p-phenylene) polymer.

3-HGN may be converted into a diaminopyridine by a process in which 3-HGN is reacted with ammonia or an ammonium donor such as an aliphatic, cyclic or aromatic amine, including amines such as n-butylamine, benzylamine, piperazine and aniline. The reaction is carried out in a solvent such as an alcohol at a temperature of 100-200° C., with the preferable use of a transition metal catalyst such as copper, cobalt, manganese or zinc salt. A process similar to the foregoing is described in U.S. Pat. No. 5,939,553.

A diaminopyridine (and thus ultimately 3-HGN as its precursor) may be converted into a polyamide oligomer or polymer by reaction with a diacid (or diacid halide) in a process in which, for example, the polymerization takes place in solution in an organic compound that is liquid under the conditions of the reaction, is a solvent for both the diacid(halide) and the diaminopyridine, and has a swelling or partial salvation action on the polymeric product. The reaction may be effected at moderate temperatures, e.g. under 100° C., and is preferably effected in the presence of an acid acceptor that is also soluble in the chosen solvent. Suitable solvents include methyl ethyl ketone, acetonitrile, N,N-dimethylacetamide dimethyl formamide containing 5% lithium chloride, and N-methylpyrrolidone containing a quaternary ammonium chloride such as methyl tri-n-butyl ammonium chloride or methyl-tri-n-propyl ammonium chloride. Combination of the reactant components causes generation of considerable heat and the agitation, also, results in generation of heat energy. For that reason, the solvent system and other materials are cooled at all times during the process when cooling is necessary to maintain the desired temperature. Processes similar to the foregoing are described in U.S. Pat. No. 3,554,966; U.S. Pat. No. 4,737,571; and CA 2,355,316.

A diaminopyridine (and thus ultimately 3-HGN as its precursor) may also be converted into a polyamide oligomer or polymer by reaction with a diacid (or diacid halide) in a process in which, for example, a solution of the diaminopyridine in a solvent may be contacted in the presence of an acid acceptor with a solution of a diacid or diacid halide, such as a diacid chloride, in a second solvent that is immiscible with the first to effect polymerization at the interface of the two phases. The diaminopyridine may, for example, be dissolved or dispersed in a water containing base with the base being used in sufficient quantities to neutralize the acid generated during polymerization. Sodium hydroxide may be used as the acid acceptor. Preferred solvents for the diacid(halide) are tetrachloroethylene, methylenechloride, naphtha and chloroform. The solvent for the diacid(halide) should be a relative non-solvent for the amide reaction product, and be relatively immiscible in the amine solvent. A preferred threshold of immiscibility is as follows: an organic solvent should be soluble in the amine solvent not more than between 0.01 weight percent and 1.0 weight percent. The diaminopyridine, base and water are added together and vigorously stirred. High shearing action of the stirrer is important. The solution of acid chloride is added to the aqueous slurry. Contacting is generally carried out at from 0° C. to 60° C., for example, for from about 1 second to 10 minutes, and preferably from 5 seconds to 5 minutes at room temperature. Polymerization occurs rapidly. Processes similar to the foregoing are described in U.S. Pat. No. 3,554,966 and U.S. Pat. No. 5,693,227.

A diaminopyridine (and thus ultimately 3-HGN as its precursor) may also be converted into a polyimide oligomer or polymer by reaction with a tetraacid (or halide derivative thereof) or a dianhydride in a process in which each reagent (typically in equimolar amounts) is dissolved in a common solvent, and the mixture is heated to a temperature in the range of 100˜250° C. until the product has a viscosity in the range of 0.1˜2 dL/g. Suitable acids or anhydrides include benzhydrol 3,3′,4,4′-tetracarboxylic acid, 1,4-bis(2,3-dicarboxyphenoxy) benzene dianhydride, and 3,3′,4,4′-benzophenone tetracarboxylic acid dianhydride. Suitable solvents include cresol, xylenol, diethyleneglycol diether, gamma-butyrolactone and tetramethylenesulfone. Alternatively, a polyamide-acid product may be recovered from the reaction mixture and advanced to a polyimide by heating with a dehydrating agent such as a mixture of acetic anhydride and beta picoline. Processes similar to the foregoing are described in U.S. Pat. No. 4,153,783; U.S. Pat. No. 4,736,015; and U.S. Pat. No. 5,061,784.

A diaminopyridine (and thus ultimately 3-HGN as its precursor) may also be converted into a polyurea oligomer or polymer by reaction with a polyisocyanate, representative examples of which include toluene diisocyanate; methylene bis(phenyl isocyanates); hexamethylene diisocycanates; phenylene diisocyanates. The reaction may be run in solution, such as by dissolving both reagents in a mixture of tetramethylene sulfone and chloroform with vigorous stirring at ambient temperature. The product can be worked up by separation with water, or acetone and water, and then dried in a vacuum oven. Processes similar to the foregoing are described in U.S. Pat. No. 4,451,642 and Kumar, Macromolecules 17, 2463 (1984). The polyurea forming reaction may also be run under interfacial conditions, such as by dissolving the diaminopyridine in an aqueous liquid, usually with an acid acceptor or a buffer. The polyisocyanate is dissolved in an organic liquid such as benzene, toluene or cyclohexane. The polymer product forms at the interface of the two phases upon vigourous stirring. Processes similar to the foregoing are described in U.S. Pat. No. 4,110,412 and Millich and Carraher, Interfacial Syntheses, Vol. 2, Dekker, New York, 1977. A diaminopyridine may also be converted into a polyurea by reaction with phosgene, such as in an interfacial process as described in U.S. Pat. No. 2,816,879.

A diaminopyridine (and thus ultimately 3-HGN as its precursor) may also be converted into a pyridobisimidazole-2,6-diyl(2,5-dihydroxy-p-phenylene) polymer by (i) converting the diaminopyridine to a diamino dinitropyridine, (ii) converting the diamino dinitropyridine to a tetramino pyridine, and (iii) converting the tetramino pyridine to a pyridobisimidazole-2,6-diyl(2,5-dihydroxy-p-phenylene) polymer.

A diaminopyridine (and thus ultimately 3-HGN as its precursor) may be converted to a diamino dinitropyridine by contacting it with nitric acid and a solution of sulfur trioxide in oleum, as discussed in WO 97/11058. A diamino dinitropyridine may be converted to a tetramino pyridine by hydrogenation using a hydrogenation catalyst in the presence of a strong acid, and using a cosolvent such as a lower alcohol, an alkoxyalcohol, acetic acid or propionic acid, as discussed in U.S. Pat. No. 3,943,125.

A tetramino pyridine (and thus ultimately 3-HGN as its precursor) may be converted to a pyridobisimidazole-2,6-diyl(2,5-dihydroxy-p-phenylene) polymer by polymerizing a 2,5-dihydroxyterephthalic acid with the trihydrochloride-monohydrate of tetraminopyridine in strong polyphosphoric acid under slow heating above 100° C. up to about 180° C. under reduced pressure, followed by precipitation in water, as disclosed in U.S. Pat. No. 5,674,969 (which is incorporated in its entirety as a part hereof for all purposes); or by mixing the monomers at a temperature from about 50° C. to about 110° C., and then 145° C. to form an oligomer, and then reacting the oligomer at a temperature of about 160° C. to about 250° C. as disclosed in U.S. Patent Publication 2006/0287475 (which is by this reference incorporated in its entirety as a part hereof for all purposes). The pyridobisimidazole-2,6-diyl(2,5-dihydroxy-p-phenylene) polymer so produced may be, for example, a poly(1,4-(2,5-dihydroxy) phenylene-2,6-pyrido[2,3-d: 5,6-d′]bisimidazole) polymer, or a poly[(1,4-dihydrodiimidazo[4,5-b:4′,5′-e]pyridine-2,6-diyl) (2,5-dihydroxy-1,4-phenylene)]polymer. The pyridobisimidazole portion thereof may, however, be replaced by any or more of a benzobisimidazole, benzobisthiazole, benzobisoxazole, pyridobisthiazole and a pyridobisoxazole; and the 2,5-dihydroxy-p-phenylene portion thereof may be replace the derivative of one or more of isophthalic acid, terephthalic acid, 2,5-pyridine dicarboxylic acid, 2,6-naphthalene dicarboxylic acid, 4,4′-diphenyl dicarboxylic acid, 2,6-quinoline dicarboxylic acid, and 2,6-bis(4-carboxyphenyl)pyridobisimidazole.

### EXAMPLES

The advantageous attributes and effects of the processes hereof may be seen in a series of examples as described below. The embodiments of these processes on which the examples are based are representative only, and the selection of those embodiments to illustrate the invention does not indicate that arrangements, approaches, components, regimes, reactants, steps, techniques, configurations, designs or protocols not described in these examples are not suitable for practicing these processes, or that subject matter not described in these examples is excluded from the scope of the appended claims and equivalents thereof.

The following materials were used in the examples. All commercial reagents were used as received. Allyl cyanide (98% purity), sodium cyanide (97% purity), and m-chloroperbenzoic acid (77% purity) were obtained from the Aldrich Chemical Company (Milwaukee, Wis., USA).

The meaning of abbreviations used in the examples is as follows: “g” means gram(s), “h” means hour(s), “mCBA” means m-chlorobenzoic acid, “mCPBA” means m-chloroperbenzoic acid, “MeOD” means deuterated methanol, “mL” means milliliter(s), “mmol” means millimole(s), “NaCN” means sodium cyanide, and “NMR” means nuclear magnetic resonance spectroscopy. The term “brine” as used herein denotes a saturated solution of sodium chloride in water.

### Example 1

1(a). Allyl cyanide epoxide for use as set forth below to make 3-HGN was prepared as follows: To a solution of allyl cyanide (6.0 mL, 74.587 mmol) in dichloromethane (100.0 mL) was added m-chloroperbenzoic acid (5.00 g of 77% mCPBA, 22.310 mmol) and the reaction mixture was stirred overnight. The next day, another 5 g of mCPBA was added, and the process was repeated for a total of 35 g of mCPBA added over seven days. Thereafter, the excess mCPBA was reduced to mCBA by adding saturated aqueous sodium hydrosulfite (NaHSO₃) solution (50.0 mL) and then diluted with water. The layers were then separated. The organic layer was extracted with saturated aqueous NaHCO₃ solution (5×100 mL) until the mCBA was removed. The organic extract was filtered through a cotton plug, and concentrated, to obtain 4.29 g (79.4%) of pure allyl cyanide epoxide.

1(b). To a cooled (0° C.) solution of NaCN (0.123 g, 2.50 mmol) in water (1.00 mL) was added concentrated sulfuric acid so that the resulting pH of the solution was about 8. Allyl cyanide epoxide (0.166 g, 2.00 mmol) prepared as set forth above in 1(a) was then added dropwise, as a solution in water (1.00 mL). The ice bath was then removed and the mixture was allowed to reach room temperature. After one hour, a 0.10 mL sample of the reaction mixture was taken, quenched with saturated aqueous NaHCO₃ solution, and extracted with ethyl acetate. Solvent was removed in vacuo, and the residue dissolved in MeOD for the NMR spectroscopic analysis. Sampling of the reaction mixture was repeated every hour for a total of four hours. The reaction was greater than 90% complete within four hours. The ratio of 3-HGN to allyl alcohol was about 60 to 1.

### Example 2

To a cooled (0° C.) solution of NaCN (0.123 g, 2.50 mmol) in water (1.00 mL) was added concentrated sulfuric acid so that the resulting pH of the solution was about 8. Allyl cyanide epoxide (0.166 g, 2.00 mmol), prepared as set forth in 1(a) above, was then added dropwise as a solution in water (1.00 mL). The ice bath was then removed and the mixture was allowed to reach room temperature over 4 h. The mixture was then partitioned between tetrahydrofuran and brine, and the layers were separated. The water layer was extracted with tetrahydrofuran four times (4×10 mL), and the organic extracts were dried over Na₂SO₄ and concentrated. 0.128 g of 3-hydroxyglutaronitrile was obtained (81% yield).

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value.

In this specification, unless explicitly stated otherwise or indicated to the contrary by the context of usage, amounts, sizes, ranges, formulations, parameters, and other quantities and characteristics recited herein, particularly when modified by the term “about”, may but need not be exact, and may also be approximate and/or larger or smaller (as desired) than stated, reflecting tolerances, conversion factors, rounding off, measurement error and the like, as well as the inclusion within a stated value of those values outside it that have, within the context of this invention, functional and/or operable equivalence to the stated value.

What is claimed is:

1. A process for preparing 3-hydroxyglutaronitrile comprising
(a) epoxidizing a solution of allyl cyanide to form allyl cyanide epoxide, which is represented by the structure of Formula I as follows: 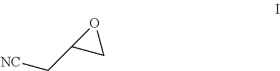
and
(b) contacting the allyl cyanide epoxide with an aqueous solution of a cyanide source that has a pH in the range of about 8 to about 10.

2. A process according to claim 1 further comprising a step of contacting m-chloroperbenzoic acid with allyl cyanide to form allyl cyanide epoxide.

3. A process according to claim 2 further comprising a step of isolating the allyl cyanide epoxide.

4. A process according to claim 1 wherein the aqueous solution of cyanide source has a pH of about 8.0 or more and yet about 9.0 or less.

5. A process according to claim 1 further comprising admixing acid with an aqueous solution of cyanide source to provide the aqueous solution of a cyanide source that has a pH in the range of about 8 to about 10.

6. A process according to claim 1 wherein the cyanide source comprises an alkali cyanide, trimethylsilyl cyanide or acetone cyanohydrin.

7. A process according to claim 1 wherein the cyanide source comprises NaCN or KCN.

8. A process according to claim 1 wherein the cyanide source contains about 1 to about 1.5 moles of cyanide per mole of allyl cyanide epoxide.

9. A process according to claim 1 wherein the temperature of the aqueous solution of cyanide source is in the range of about 0 to about 25° C. at the time the allyl epoxide is contacted therewith.

10. A process according to claim 1 wherein 3-hydroxyglutaronitrile is recovered from the reaction mixture.

11. A process according to claim 1 wherein 3-hydroxyglutaronitrile is subjected, with or without recovery from the reaction mixture, to conversion to a compound, oligomer or polymer.

12. A process according to claim 1 further comprising a step of subjecting the 3-hydroxyglutaronitrile to one or more reactions to prepare therefrom a compound, oligomer or polymer.

13. A process according to claim 12 wherein a compound prepared comprises diamino pyridine.

14. A process according to claim 13 wherein preparation of a polymer comprises conversion of diamino pyridine to a polymer.

15. A process according to claim 12 wherein a polymer prepared comprises a pyridobisimidazole-2,6-diyl(2,5-dihydroxy-p-phenylene) polymer, or a poly[(1,4-dihydrodiimidazo[4,5-b:4′,5′-e]pyridine-2,6-diyl) (2,5-dihydroxy-1,4-phenylene)]polymer.
